(19) United States
(12) Patent Application Publication
(10) Pub. No.: US 2023/0014925 A1
(43) Pub. Date: Jan. 19, 2023

(54) SURGICAL TOOL

(71) Applicant: RIVERFIELD INC., Tokyo (JP)

(72) Inventor: Koki SHINDO, Tokyo (JP)

(73) Assignee: RIVERFIELD INC., Tokyo (JP)

(21) Appl. No.: 17/952,874

(22) Filed: Sep. 26, 2022

Related U.S. Application Data
(63) Continuation of application No. PCT/JP2020/014157, filed on Mar. 27, 2020.

(51) Int. Cl. A61B 34/30; A61B 17/29

(52) U.S. Cl. CPC A61B 34/30; A61B 17/29; A61B 34/71

(57) ABSTRACT

A surgical tool mounted to a surgical robot includes a treatment portion including a movable part that may be displaced, a main body including a slider that generates a driving force to displace the movable part, the slider generating the driving force through reciprocating motion along a linear direction, and a manual releasing tool that, when inserted into the main body, may displace the movable part, the manual releasing tool being detachably insertable into the main body.

### CROSS REFERENCE TO RELATED APPLICATION

This U.S. Application is a continuation application of International Application No. PCT/JP2020/014157 on filed Mar. 27, 2020, in the Japanese Patent Office, the contents of which being incorporated by reference herein in its entirety.

### BACKGROUND

The present disclosure relates to a surgical tool mounted to a surgical robot.

A surgical robot may include a surgical tool that is inserted into a patient to perform a surgical operation. However, when the power supply is stopped in a state in which the surgical tool grips a tissue of a patient, it is difficult to continue a surgical operation or to discontinue the surgical operation.

### SUMMARY

It is an aspect to provide a surgical tool that is mounted to a surgical robot, the surgical tool being capable of dealing with a case where power supply is stopped.

According to an aspect of one or more embodiments, there is provided a surgical tool mounted to a surgical robot, the surgical tool comprising a treatment portion used in surgical treatment, the treatment portion including a movable part that is configured to be displaced; a slider that generates a driving force to displace the movable part, the slider generating the driving force through reciprocating motion along a linear direction; and a manual releasing tool that is configured to displace the movable part, the manual releasing tool being detachably insertable into a main body in which the slider is accommodated.

According to another aspect of one or more embodiments, there is provided a surgical tool mounted to a surgical robot, the surgical tool comprising a treatment portion including a movable part that is configured to be displaced; a main body including a slider that generates a driving force to displace the movable part, the slider generating the driving force through reciprocating motion along a linear direction; and a manual releasing tool that is configured to displace the movable part, the manual releasing tool being detachably insertable into the main body.

According to yet aspect of one or more embodiments, there is provided a surgical tool comprising a forceps including a gripper; a main body including a slider that reciprocates to displace the gripper; and a manual releasing tool that is detachably insertable into the main body, wherein when power to the surgical tool is stopped, the manual releasing tool is inserted into the main body to manually displace the gripper.

### BRIEF DESCRIPTION OF THE DRAWINGS

The above and/or other aspects will become apparent and more readily appreciated from the following description of exemplary embodiments, taken in conjunction with the accompanying drawings in which

FIG. 1 is an external diagram of a surgical robot according to some embodiments;

FIG. 2 is a block diagram of the surgical robot according to some embodiments;

FIG. 3 is a diagram showing a distal end side of a robot arm and a surgical tool according to some embodiments;

FIG. 4 is a diagram showing a distal end side of the surgical tool according to some embodiments;

FIG. 5 is a diagram showing a structure of a main body according to some embodiments;

FIG. 6 is a diagram showing an appearance of the main body according to some embodiments;

FIG. 7 is a diagram showing the structure of the main body according to some embodiments;

FIG. 8 is a diagram showing the structure of the main body according to some embodiments; and

FIG. 9 is a diagram showing an appearance of a main body according to some embodiments.

### DETAILED DESCRIPTION

A medical support arm device may be capable of continuing a surgical operation even when power supply to the medical support arm device is stopped during the surgical operation.

Among those surgical tools mounted to a surgical robot, for example, a treatment instrument, such as forceps, is provided with a treatment portion such as a hand part (a gripper) on a distal end side. The treatment portion is provided with at least one movable part.

The movable part can be directly or indirectly displaced by an actuator using air pressure or electric power. Because of this configuration, if a supply of air pressure, or, power supply is stopped, and then the actuator is stopped, a state of the movable part is retained as the state is when the actuator is stopped.

As described above, for example, when the power supply is stopped in a state in which the gripper of forceps (a surgical tool) grips a tissue of a patient, it is difficult to continue the surgical operation or to discontinue the surgical operation.

The present disclosure provides an example of a surgical tool that is mounted to the surgical robot, the surgical tool being capable of dealing with a case where power supply is stopped.

According to some embodiments, a surgical tool mounted to a surgical robot may include a treatment portion used in surgical treatment, the treatment portion including at least one movable part that can be displaced; a slider that generates a driving force to displace the movable part, the slider generating the driving force through reciprocating motion along a linear direction; and a manual releasing tool for displacing the movable part, the manual releasing tool being detachably mountable to a main body at which the slider is accommodated.

Thus, in the surgical tool, when a user mounts the manual releasing tool to the main body, the user can displace the movable part by manual operation through the manual releasing tool. Therefore, the surgical tool makes it possible, for example, to continue the surgical operation or to discontinue the surgical operation to deal even with a case where the power supply is stopped.

In this specification, for at least a member or portion described with a reference numeral affixed thereto, there is at least one in number unless specified as “one” or the like. In other words, the member or portion may be two or more in number unless specified as “one”. The surgical robot and the surgical tool shown in the present disclosure comprises at least components such as members or portions described with reference numerals affixed thereto, and structural portions shown in the drawings.

### 1. Overview of Surgical Robot

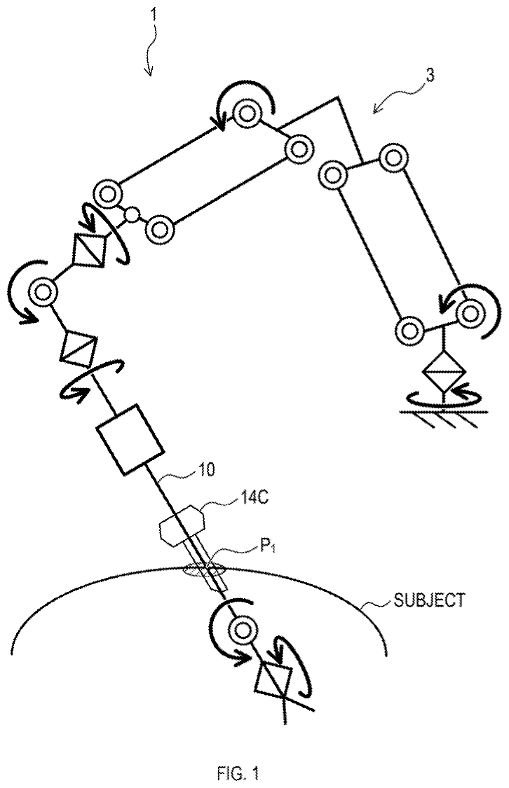

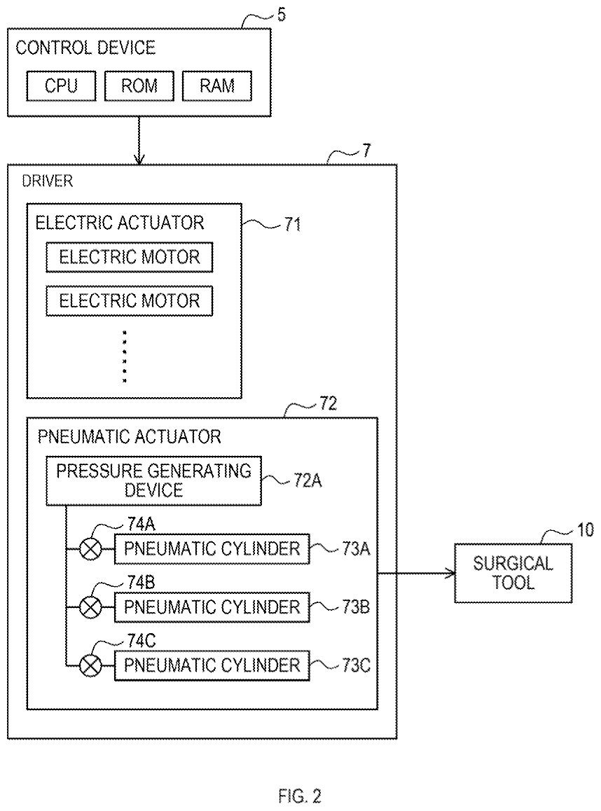

FIG. 1 is an external diagram of a surgical robot according to some embodiments. FIG. 2 is a block diagram of the surgical robot according to some embodiments. In some embodiments, a surgical robot may be used in an endoscopic surgical operation. A surgical robot 1 shown in FIG. 1 is an example of a surgical robot for use in endoscopic surgical operation. The surgical robot 1 comprises, as shown in FIG. 2, a robot arm 3, a control device 5, a driver 7 and a surgical tool 10.

### Robot Arm

As shown in FIG. 1, the robot arm 3 is an example of an arm device that can displace the surgical tool 10 in a state in which the surgical tool 10 is held. Specifically, the robot arm 3 is configured by a link mechanism having two or more joints.

### Driver

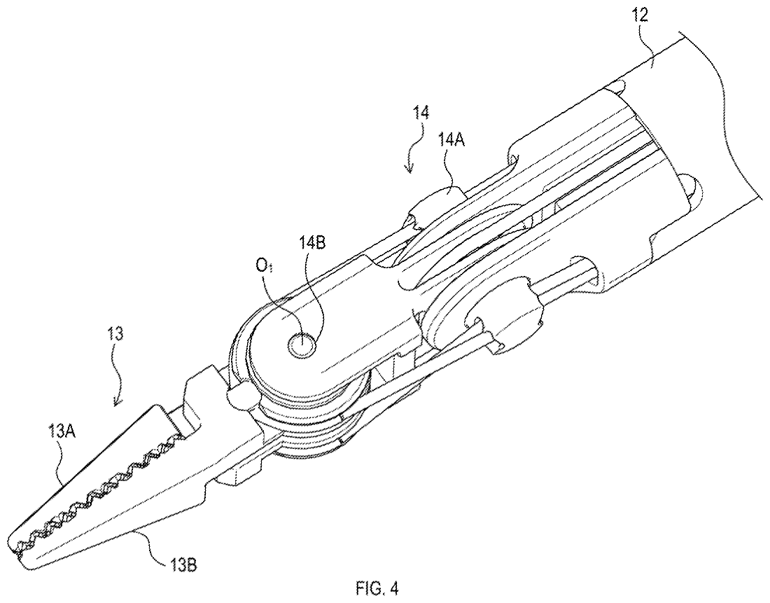

As shown in FIG. 4, the driver 7 is an example of a driving part for driving movable parts 13A and 13B of the robot arm 3 and the surgical tool 10. In some embodiments, the movable parts 13A and 13B may be hands of a gripper tool. In some embodiments, the driver 7 may comprise an electric actuator 71 and a pneumatic actuator 72, as shown in FIG. 2.

The electric actuator 71 is configured by at least one electric motor and drives each of the joints. In some embodiments, each joint is provided with an electric motor. Thus, each electric motor is arranged at the robot arm 3.

As shown in FIG. 4, the pneumatic actuator 72 operates at least the movable parts 13A and 13B. As shown in FIG. 2, the pneumatic actuator 72 comprises pneumatic cylinders 73A to 73C, a pressure generating device 72A, and the same number of controller solenoid valves 74A to 74C as the pneumatic cylinders. While the example illustrated in FIG. 2 shows three pneumatic cylinders 73A to 73C and three solenoid valves 74A to 74C, this is only an example and, in some embodiments, the pneumatic actuator 72 may include one or more pneumatic cylinders and one or more solenoid valves.

The pressure generating device 72A is a pressure supply source that supplies compressed air to the pneumatic cylinders 73A to 73C. Each of the pneumatic cylinders 73A to 73C is operated by receiving an air pressure. Each of the controller solenoid valves 74A to 74C controls the air pressure supplied to each of the pneumatic cylinders 73A to 73C, to thereby control operations of the corresponding pneumatic cylinders 73A to 73C.

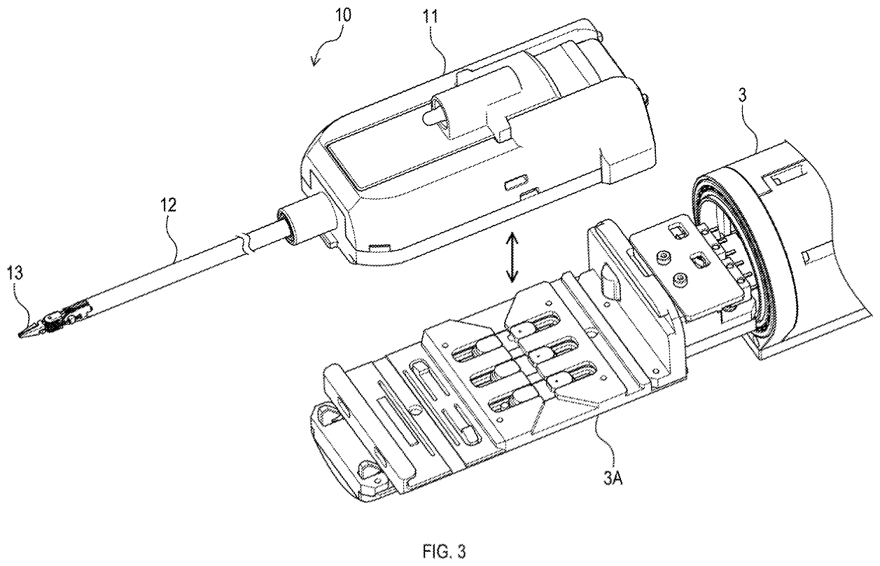

In some embodiments, at least the pneumatic cylinders 73A to 73C are provided at the robot arm 3. As shown in FIG. 3, the surgical tool 10 is detachably mounted to mounted portion 3A provided at a distal end of the robot arm 3.

### Control Device

The control device 5 controls operations of the driver 7, more specifically the electric actuator 71, the pneumatic actuator 72, and the like. In some embodiments, the control device 5 may include a central processing unit (CPU), a read only memory (ROM) and a random access memory (RAM). The CPU reads program code from the ROM and/or the RAM and executes the program code to perform the functions of the control device described herein. In some embodiments, the control device may include one or more processors and one or more memories, or hardware control logic. In some embodiments, the one or more processors may be a microprocessor, an application specific integrated circuit (ASIC), or a microcontroller that reads program code from the one or more memories and executes the program code to perform the functions of the control device 5 described herein. In some embodiments, the hardware control logic may be arranged to perform the functions of the control device 5 described herein. More specifically, the control device 5 controls the controller solenoid valves 74A to 74C. The control device 5 receives a command signal output from a master-side input operation device (not shown) and operates the driver 7 according to the command signal.

At this time, as shown in FIG. 1, the control device 5 controls the operation of the electric actuator 71 so that a portion of the surgical tool 10 corresponding to an incision position P1 of a subject is immovable. The incision position P1 is a site into which a trocar 14C as shown in FIG. 1 is inserted during endoscopic surgical operation.

The trocar 14C is a cylindrical member. A trocar is also referred to as a trochar. The trocar may be also described a “cannula”. An operator inserts the surgical tool 10, such as an endoscope or forceps, into a body of the subject through the trocar 14C, and performs a surgical operation while looking at an image captured by the endoscope.

### 2. Surgical Tool

### 2.1 Overview of Surgical Tool

The surgical tool 10 comprises, as shown in FIG. 3, a main body 11, an insertion part 12, and a treatment portion 13. The main body 11 is a part that is detachably mountable to the robot arm 3, more specifically to mounted portion 3A.

The insertion part 12 is a tubular part extending from the main body 11. The insertion part 12 is a part that is inserted into a body through the trocar 14C and formed from a material with a high bending rigidity, such as a metal.

At a distal end portion of the insertion part 12, the treatment portion 13 is provided. As shown in FIG. 4, the treatment portion 13 comprises movable parts 13A and 13B. While the example illustrated in FIG. 4 shows two movable parts 13A and 13B, this is only an example and, in some embodiments, the treatment portion 13 may include one or more movable parts.

The movable parts 13A and 13B are parts displaceable with respect to the insertion part 12. Specifically, the surgical tool 10 according to some embodiments is forceps. In other words, the treatment portion 13 according to some embodiments is a gripper that can grip a portion to be treated, a suturing needle, or the like.

The two movable parts 13A and 13B can be rotationally displaced or pivoted around a central axis O1 between a closed position (see, FIG. 4) and an open position in which their distal ends are separated. Thus, the treatment portion 13 can grip a target site or the like.

The treatment portion 13 according to some embodiments is connected to a distal end of the insertion part 12 via the joint 14. The joint 14 is a mechanism for changing an angle of the treatment portion 13. Specifically, the joint 14 comprises rotational shafts 14A and 14B. While the example illustrated in FIG. 4 shows two rotational shafts 14A and 14B, this is only an example and, in some embodiments, the joint 14 may include one or more rotational shafts.

The joint 14 can be rotationally displaced around each of the rotational shafts 14A and 14B as a central axis. A first rotational shaft 14A is a shaft perpendicular to a longitudinal direction of the insertion part 12. A second rotational shaft 14B is a shaft perpendicular to the longitudinal direction of the insertion part 12 and the first rotational shaft 14A.

### 2.2 Displacement of Movable Part

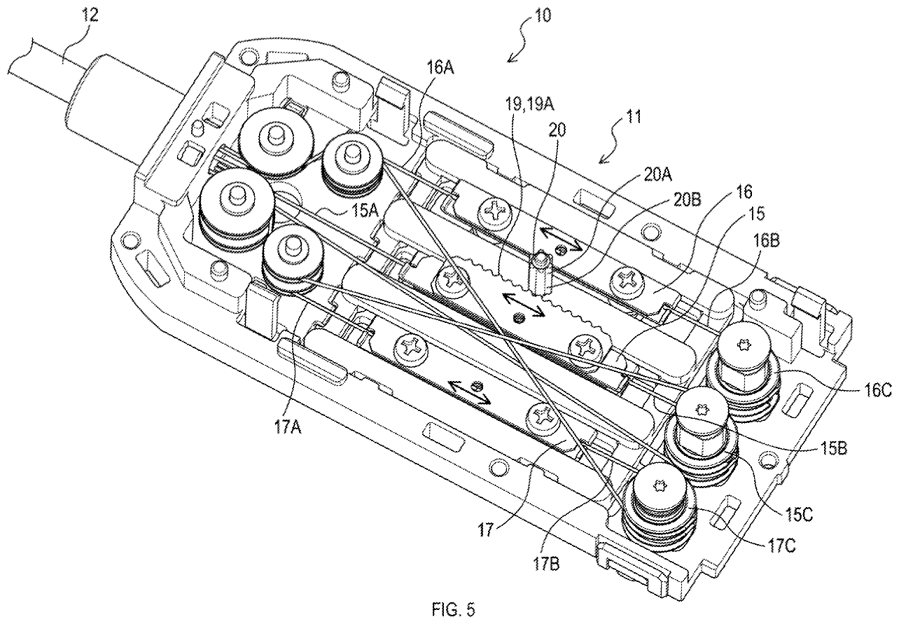

Inside the main body 11, as shown in FIG. 5, sliders 15 to 17 are accommodated. While the example illustrated in FIG. 5 shows three slides 15-17, this is only an example and, in some embodiments, the main body 11 may include one or more sliders. Each of the sliders 15 to 17 is a member that is reciprocatingly movable along a linear direction.

In some embodiments, each of the sliders 15 to 17 is displaced along a direction parallel to the longitudinal direction (specifically, an arrow direction shown in FIG. 5) of the insertion part 12. Each of the sliders 15 to 17 is displaced by receiving a driving force from the pneumatic cylinders 73A to 73C as shown in FIG. 2.

The slider 15 makes a reciprocating motion, thereby generating a driving force to displace the movable part 13A. A first end 15A of a driving wire 15W and a second end 15B of the driving wire 15W are respectively connected to a first end and a second end of the slider 15 along a displacement direction.

The slider 16 makes a reciprocating motion, thereby generating a driving force to displace the movable part 13B. A first end 16A of a driving wire 16W and a second end 16B of the driving wire 16W are respectively connected to a first end and a second end of the slider 16 along a displacement direction.

The driving wire 15W is directly or indirectly connected to the movable part 13A. Therefore, when the slider 15 is displaced, the movable part 13A is displaced in conjunction with the displacement. Specifically, when the slider 15 makes the reciprocating motion, the movable part 13A of the treatment portion 13 configuring the gripper is displaced around the second rotational shaft 14B in conjunction with the reciprocating motion.

The driving wire 16W is directly or indirectly connected to the movable part 13B. Therefore, when the slider 16 is displaced, the movable part 13B is displaced in conjunction with the displacement. Specifically, when the slider 16 makes the reciprocating motion, the movable part 13B of the treatment portion 13 configuring the gripper is displaced around the second rotational shaft 14B in conjunction with the reciprocating motion.

In a case in which turning directions of the movable part 13A and the movable part 13B are opposite to each other, the treatment portion 13 opens and closes. In a case in which the turning directions of the movable part 13A and the movable part 13B are the same, the treatment portion 13 is displaced around the second rotational shaft 14B.

The slider 17 generates a driving force to operate the joint 14. A first end 17A of a driving wire 17W and a second end 17B of the driving wire 17W are respectively connected to a first end and a second end of the slider 17 along a displacement direction.

The driving wire 17W displaces the treatment portion 13 and the second rotational shaft 14B around the first rotational shaft 14A. Thus, the joint 14 is operated in conjunction with displacement of the sliders 15, 16 and 17.

A pulley 15C is a fixed pulley that redirects the driving wire 15W. A pulley 16C is a fixed pulley that redirects the driving wire 16W. A pulley 17C is a fixed pulley that redirects the driving wire 17W.

### 2.3 Manual Release of Treatment Portion (Gripper)

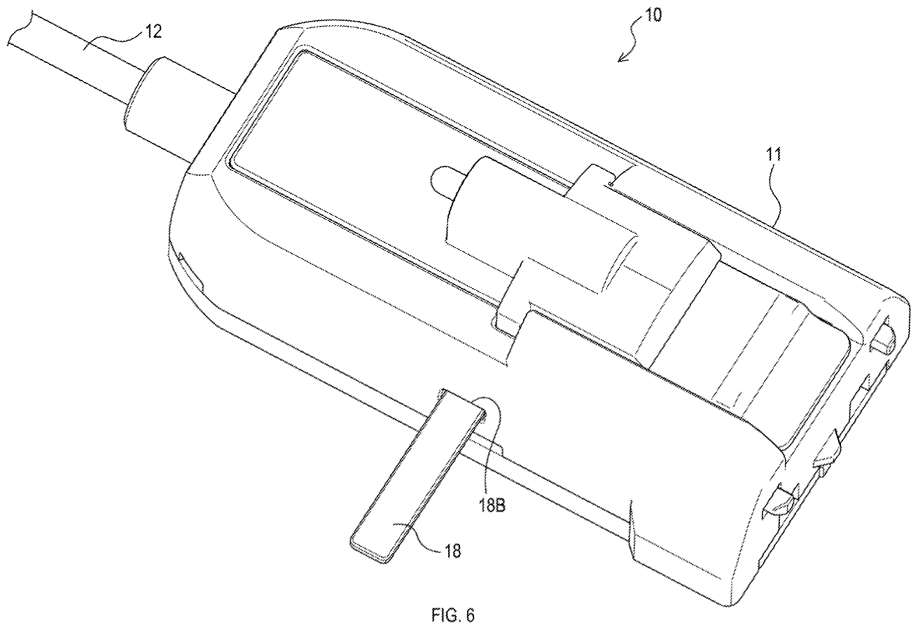

As shown in FIG. 6, the surgical tool 10 comprises a manual releasing tool 18. The manual releasing tool 18 is a member for displacing the movable part 13A and is detachable with respect to the main body 11.

FIG. 6 shows a state in which the manual releasing tool 18 is mounted to the main body 11. FIG. 3 shows a state in which the manual releasing tool 18 is not mounted. In the state in which the manual releasing tool 18 is not mounted, the manual releasing tool 18 is held by, for example, the robot arm 3.

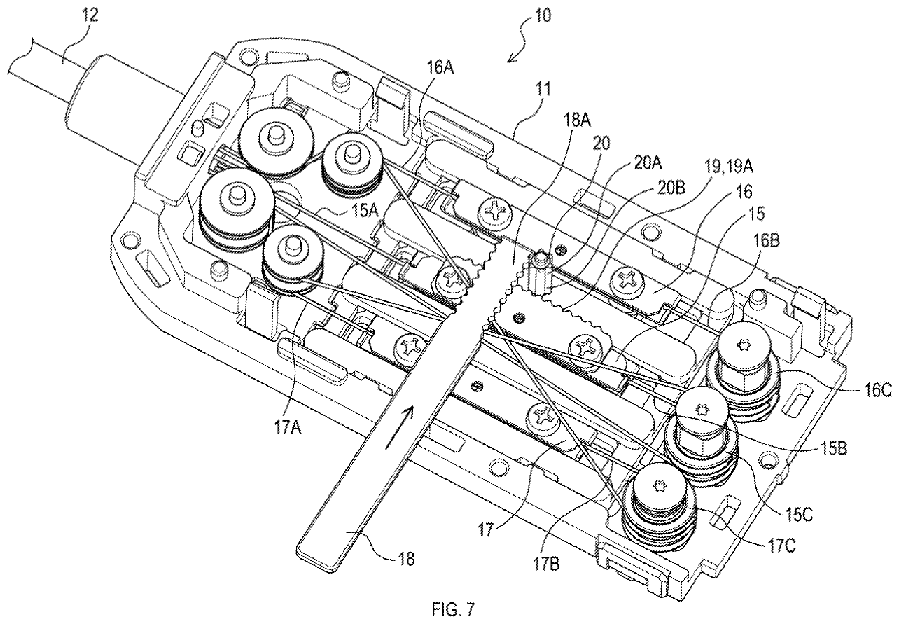

On the slider 15, as shown in FIG. 5, a receiver 19 is provided. The receiver 19 is a part that directly or indirectly receives a driving force from the manual releasing tool 18. In some embodiments, the receiver 19 indirectly receives the driving force from the manual releasing tool 18, as shown in FIG. 7.

The receiver 19 according to some embodiments is configured by a rack portion 19A that extends along a direction parallel to a sliding direction of the slider 15. Here, a rack is a sector gear whose curvature radius is infinite. Hereinafter, the receiver 19 will be also referred to as the rack portion 19A. In other words, the receiver 19 comprises the rack portion 19A.

The manual releasing tool 18 according to some embodiments is mounted to the main body 11 so as to be displaceable along a direction intersecting the displacement direction of the slider 15. In some embodiments, the manual releasing tool 18 may be mounted perpendicular to the main body 11 so as to be displaceable along a direction perpendicular to the displacement direction of the slider 15. The manual releasing tool 18 is provided with a rack portion 18A on its distal end side in a direction of insertion.

The main body 11 is provided with a pinion member 20A that is engageable with the rack portion 18A and a pinion member 20B that is engageable with the rack portion 19A. The pinion member 20A and the pinion member 20B are integrated together in such a manner that the respective rotational axes of the pinion member 20A and the pinion member 20B coincide. Hereinafter, the two integrated pinion members 20A and 20B will be referred to as a pinion 20. In other words, the pinion 20 comprises the pinion member 20A and the pinion member 20B. While two pinion members 20A and 20B are illustrated in FIG. 7, this is only an example and, in some embodiments, a single pinion member may be provided.

Thus, when the manual releasing tool 18 is displaced in a state in which the manual releasing tool 18 and the pinion member 20A are engaged with each other, the pinion member 20B is rotated, whereby the rotation is transmitted to the rack portion 19A.

In the pinion 20, a position of the rotational axis is immovable relative to the main body 11. Thus, when the manual releasing tool 18 is displaced in the state in which the manual releasing tool 18 and the pinion 20 are engaged with each other, the slider 15 is displaced along a linear direction by receiving a driving force from the rack portion 19A.

The pinion 20 functions as a transmitter that converts a mounting force received by the manual releasing tool 18 when the manual releasing tool 18 is mounted to the main body 11, and transmits the converted mounting force to the receiver 19. In some embodiments, when the manual releasing tool 18 is displaced in the direction of insertion (in other words, a direction of an arrow in FIG. 7), the movable part 13A is displaced so as to open the treatment portion 13.

The mounting force corresponds to one example of an operating force.

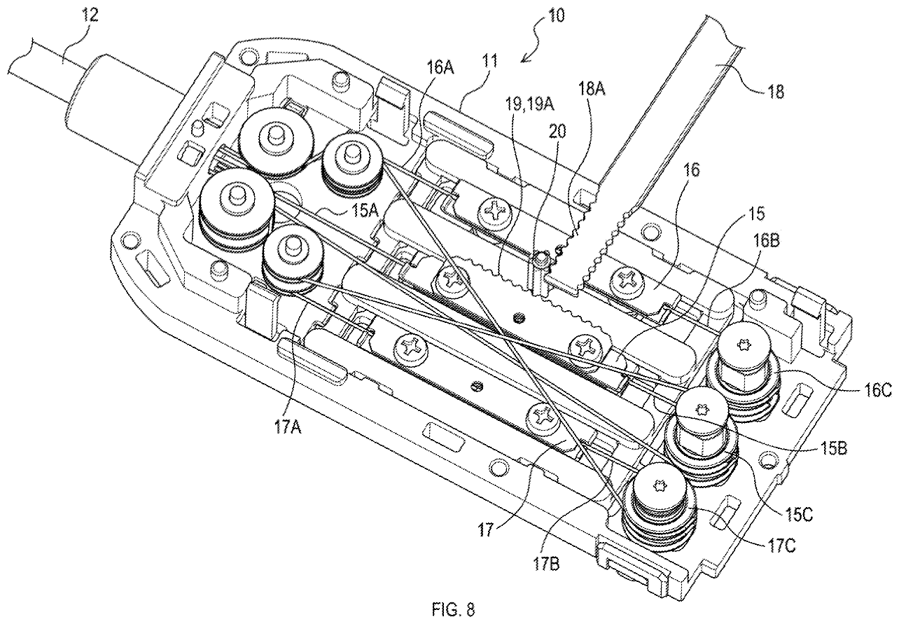

As shown in FIG. 8, the surgical tool 10 according to some embodiments allows the user to insert the manual releasing tool 18 into the main body 11 from a position different from a position shown in FIG. 7. The main body 11 according to some embodiments is provided with a receiving portion 18B configured such that the manual releasing tool 18 can be received into the receiving portion 18B as shown in FIG. 6. While one receiving portion is illustrated in FIG. 6, this is only an example and, in some embodiments, the main body 11 may comprise two or more receiving portions. In some embodiments, the receiving portion 18 may be a slot or a hole in the main body 11.

In a configuration in which two or more receiving portions 18B are provided in the main body 11, a first mounted portion 18B is arranged on a first side of the main body 11, and a second mounted portion 18B is arranged on a second side of the main body 11. The second mounted portion 18B is arranged at a position rotated by 180 degrees with respect to the first mounted portion 18B about the rotational axis of the pinion 20.

### 3. Features of Surgical Robot (Particularly, Surgical Tool)

The surgical tool 10 according to some embodiments comprises the manual releasing tool 18 for displacing the movable part 13A, the manual releasing tool 18 being detachably mountable to the main body 11.

Thus, in the surgical tool 10 according to some embodiments, when the user inserts the manual releasing tool 18 to the main body 11, the user can displace the movable part 13A by manual operation through the manual releasing tool 18. Therefore, the surgical tool makes it possible, for example, to continue the surgical operation or to discontinue the surgical operation to deal even with a case where the power supply is stopped.

The slider 15 is provided with the receiver 19 that directly or indirectly receives the driving force from the manual releasing tool 18. The slider 15 can be displaced along the linear direction by receiving the driving force from the receiver 19. Thus, the user can reliably displace the movable part 13A by manual operation through the manual releasing tool 18.

In some embodiments, when the manual releasing tool 18 is inserted into the main body 11, the rack portion 18A and the pinion member 20A are engaged with each other, whereby the slider 15 is displaced. Specifically, in some embodiments, the mounting force received by the manual releasing tool 18 is converted into a force to displace the slider 15 at the transmitter and the receiver 19. The pinion 20 corresponds to one example of the transmitter.

Therefore, the user can displace the movable part 13A by only inserting the manual releasing tool 18 to the main body 11 in an inserting manner. This configuration and operation makes it possible for the user to quickly and reliably open the treatment portion 13. The treatment portion 13 corresponds to one example of the gripper.

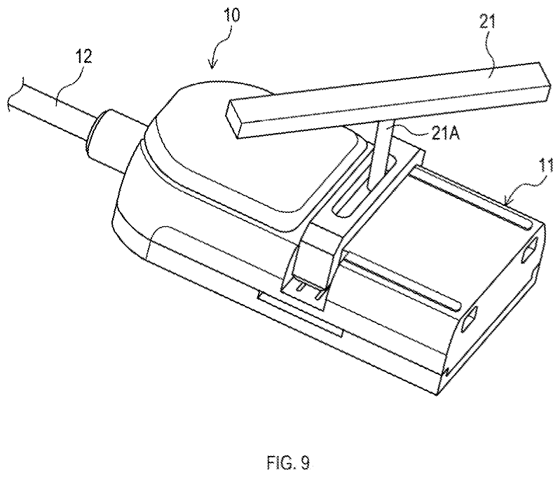

FIG. 9 is a diagram showing an appearance of a main body according to some embodiments. As shown in FIG. 9, a manual releasing tool 21 according to some embodiments is a member connectable with the pinion member 20B. For this reason, in some embodiments, the rack portion 18A and the pinion member 20A may be omitted, and the pinion member 20B is included in the pinion 20. In other words, a single pinion member may be provided, and is referred to as the pinion member 20B for convenience.

The pinion member 20B is provided with a connecting member (not shown) that is detachably connectable with an axial member 21A shown in FIG. 9 of the manual releasing tool 21. The user can displace the movable part 13A by rotating the manual releasing tool 21 in a state in which the axial member 21A of the manual releasing tool 21 is connected to the connecting member.

In some embodiments, the transmitter (i.e., the pinion 20) may be omitted, and a pinion member may be configured to be arranged in the axial member 21A of the manual releasing tool 21. According to this configuration, when the manual releasing tool 21 is inserted into the main body 11, and the pinion member of the axial member 21A is engaged with the rack portion 19A, the slider 15 goes into a state in which it is displaceable by a manual operation of turning the manual releasing tool 21.

### Other Embodiments

The sliders 15 to 17 each is configured to make the reciprocating motion along a direction parallel to the longitudinal direction of the insertion part 12. However, embodiments are not limited to this. For example, in some embodiments, the sliders 15 to 17 each may be configured to make the reciprocating motion along a direction intersecting the longitudinal direction of the insertion part 12.

The pinion member 20B and the rack portion 19A are provided, and the pinion member 20B and the rack portion 19A are configured to be engaged with each other, whereby operating forces of the manual releasing tools 18 and 21 are transmitted to the slider 15. However, embodiments are not limited to this. For example, in some embodiments, the pinion member 20B and the rack portion 19A may be omitted, and a friction force generated at a contact area between the transmitter and the slider 15 is used to transmit the operating forces to the slider 15.

The operating forces of the manual releasing tools 18 and 21 are configured to be transmitted to the slider 15 through the transmitter. However, embodiments are not limited to this. For example, in some embodiments, the manual releasing tool 18 or the manual releasing tool 21 may be configured to be detachably connectable to the slider 15.

In this configuration, the slider 15 may be configured to be displaced by the user displacing the manual releasing tool 18 or the manual releasing tool 21 along the sliding direction in a state in which the manual releasing tool 18 or the manual releasing tool 21 is connected to the slider 15.

The movable part 13A is configured to be displaced by displacing the slider 15 with the manual releasing tool 18 or the manual releasing tool 21. However, embodiments are not limited to this. For example, in some embodiments, a member other than the slider 15 such as the driving wire 15W can be operated with the manual releasing tool 18 or the manual releasing tool 21.

Mounting directions of the manual releasing tools 18 and 21 are perpendicular to the sliding direction of the slider 15. However, embodiments are not limited to this. For example, in some embodiments, the mounting directions of the manual releasing tool 18 or the manual releasing tool 21 may be parallel to the sliding direction.

In some embodiments, two receiving portions 18B to which the manual releasing tool 18 can be inserted are provided. However, as discussed above, embodiments are not limited to this. For example, in some embodiments, one, or three or more receiving portions 18B may be provided.

The displacement of the slider 15 is configured to be transmitted to the movable part 13A through the driving wire 15W. However, embodiments are not limited to this. For example, in some embodiments, the displacement of the slider 15 may be configured to be transmitted to the movable part 13A through a push-pull wire or a geared cable.

The treatment portion 13 may be configured by a so-called gripper and is a portion separate from the joint 14. However, embodiments are not limited to this. For example, in some embodiments, the treatment portion 13 may be configured by a component other than the gripper or may be configured such that the treatment portion 13 and the joint 14 are integrated together; in other words, in some embodiments, the joint 14 can also be operated with the manual releasing tool 18 or the manual releasing tool 21.

The manual releasing tools 18 and 21 are configured to displace the slider 15. However, embodiments are not limited to this. For example, in some embodiments, the manual releasing tool 18 or the manual releasing tool 21 can displace the sliders 16 and 17, as well.

The slider 15 is configured to be operated through the manual releasing tool 18 or the manual releasing tool 21. However, embodiments are not limited to this. For example, the slider 15 may be provided with a projecting operating part and may be configured such that a distal end side of the operating part protrudes from the main body 11 enough that it can be operated by the user. In this configuration, the manual releasing tool 18 or the manual releasing tool 21 are not necessary and may be omitted.

Further, the present disclosure is not limited to the aforementioned embodiments. Accordingly, the present disclosure may be configured in combination of at least two of the aforementioned embodiments, or may be configured without some of the components illustrated in the drawings or described with reference numerals in the aforementioned embodiments.

It should be understood that the present disclosure is not limited to the above embodiments, but various other changes and modifications may be made therein without departing from the spirit and scope of the appended claims.

What is claimed is:

1. A surgical tool mounted to a surgical robot, the surgical tool comprising:
a treatment portion used in surgical treatment, the treatment portion including a movable part that is configured to be displaced;
a slider that generates a driving force to displace the movable part, the slider generating the driving force through reciprocating motion along a linear direction; and
a manual releasing tool that is configured to displace the movable part, the manual releasing tool being detachably insertable into a main body in which the slider is accommodated.

2. The surgical tool according to claim 1,
wherein the slider is provided with a receiver that directly or indirectly receives the driving force from the manual releasing tool, and
wherein the slider is displaced along the linear direction by receiving the driving force from the receiver.

3. The surgical tool according to claim 2, further comprising a transmitter arranged in the main body, the transmitter converting an insertion force received from the manual releasing tool, when the manual releasing tool is inserted into the main body, and transmitting the converted insertion force to the receiver.

4. The surgical tool according to claim 3,
wherein the manual releasing tool is configured to be inserted into the main body so as to be displaceable along a direction intersecting a displacement direction of the slider, and
wherein the transmitter includes a first pinion member that is engageable with a rack portion provided in the manual releasing tool.

5. The surgical tool according to claim 4, wherein the transmitter includes a second pinion member that is engageable with a rack portion configuring the receiver.

6. The surgical tool according to claim 2, wherein the receiver receives the driving force by rotation of the manual releasing tool that is inserted into the main body.

7. The surgical tool according to claim 6, wherein the receiver includes a rack portion that is engageable with a pinion member provided in the manual releasing tool.

8. The surgical tool according to claim 1, wherein the main body is provided with two or more receiving portions for receiving the manual releasing tool.

9. A surgical tool mounted to a surgical robot, the surgical tool comprising:
a treatment portion including a movable part that is configured to be displaced;
a main body including a slider that generates a driving force to displace the movable part, the slider generating the driving force through reciprocating motion along a linear direction; and
a manual releasing tool that is configured to displace the movable part, the manual releasing tool being detachably insertable into the main body.

10. The surgical tool according to claim 9,
wherein the slider comprises a rack that directly or indirectly receives the driving force from the manual releasing tool, and
wherein the slider is displaced along the linear direction by the driving force from the rack.

11. The surgical tool according to claim 10, wherein the main body comprises a pinion that engages with the rack when the manual releasing tool is inserted into the main body.

12. The surgical tool according to claim 11,
wherein the manual releasing tool is displaceable along a direction intersecting the linear direction,
the manual releasing tool comprises a rack at a distal end thereof, and
wherein the pinion is engageable with the rack of the manual releasing tool.

13. The surgical tool according to claim 12, wherein the pinion is engageable with the rack of the slider.

14. The surgical tool according to claim 10, wherein the rack is rotated by a rotation of the manual releasing tool that is inserted into the main body.

15. The surgical tool according to claim 14, wherein the manual releasing tool comprises a pinion, and
the rack of the slider engages with the pinion of the manual releasing tool.

16. The surgical tool according to claim 9, wherein the main body comprises two or more slots for receiving the manual releasing tool.

17. A surgical tool comprising:
a forceps including a gripper;
a main body including a slider that reciprocates to displace the gripper; and
a manual releasing tool that is detachably insertable into the main body,
wherein when power to the surgical tool is stopped, the manual releasing tool is inserted into the main body to manually displace the gripper.

18. The surgical tool according to claim 17, wherein
wherein the slider comprises a rack,
the main body comprises a pinion that is engaged with the rack of the slider,
the manual releasing tool comprises a rack at a distal end thereof, and
when the manual releasing tool is inserted in to the main body, the rack of the manual releasing tool engages with the pinion.

19. The surgical tool according to claim 17, wherein
wherein the slider comprises a rack,
the manual releasing tool comprises a pinion at a distal end thereof, and
when the manual releasing tool is inserted in to the main body, the pinion of the manual releasing tool engages with the rack.

20. A surgical robot comprising:
a robot arm;
the surgical tool according to claim 17, the surgical tool being attached to a distal end of the robot arm; and
a control device that, under the power, controls the slider to reciprocal to displace the gripper.
